(19) United States
(12) Patent Application Publication
(10) Pub. No.: US 2024/0173238 A1
(43) Pub. Date: May 30, 2024

(54) Temporary Wrinkle Softener

(71) Applicant: Fuler, LLC, Sheboygan, WI (US)

(72) Inventor: Leda Z. Fessler, Sheboygan, WI (US)

(21) Appl. No.: 18/070,994

(22) Filed: Nov. 29, 2022

(51) Int. Cl. A61K 8/64; A61K 8/98

(52) U.S. Cl. CPC A61K 8/64; A61K 8/982; A61K 2800/48; A61K 2800/524

(57) ABSTRACT

A daily wear cosmetic essence that temporarily softens the appearance of wrinkles such as the eleven-lines. The essence causes a tightening effect that lightens and flattens wrinkles. The essence is well-suited for daily wear because it may be easily self-applied, is invisible and scentless, provides near immediate results, and may be easily washed off. The composition of the essence may be primarily made of albumen, for example egg white. The albumen may act as a tightening agent that causes the tightening effect. Additional supplementary components may be added to the essence to preserve the albumen from spoiling, to thicken the essence, and to prevent flaking when applying.

### BACKGROUND OF THE INVENTION

### 1. Field of the Invention

The invention relates to a cosmetic essence worn daily to soften the appearance of wrinkles, in particular the eleven-lines. The cosmetic essence tightens the skin making the eleven-lines less visible.

### 2. Discussion of the Related Art

Currently on the market, many cosmetic products claim to reduce the appearance of aging. One such approach is reducing the visibility of wrinkles, especially wrinkles on the face. Cosmetic products attempt to treat or cover up these facial wrinkles. One set of wrinkles that is often targeted is known as the eleven-lines. The eleven-lines are two or more vertical wrinkles between the eyebrows and can extend from the forehead to the eyes. As with most wrinkles, the eleven-lines become more visible with age. Further, the eleven-line are often made more prominent when a person frowns.

Many cosmetic products that claim to reduce the appearance of the eleven-lines and other wrinkles are designed to be applied in a private environment. These products are instantly noticeable on the user's face when they are applied. Because it is apparent when these products are being worn, the products often recommend applying them at home at night. Some of these products claim to produce near immediate results while others claim results will come after repeated use.

For example, numerous patches and pads claim to treat or reduce the appearance of facial wrinkles, such as the eleven-lines. The patches and pads are designed to be placed on a wrinkled area for a temporary period. However, these patches and pads are not intended to be worn outside of a private environment because they are readily noticeable on a user's face. Similarly, certain creams, gels, and ointments claim to treat wrinkles but have a noticeable color or odor when applied on a user. Thus, these products are not meant to be worn out in public.

Another common wrinkle treatment is Botox injections. Botox injections involve injecting a toxin into muscles temporarily preventing the muscles from moving. This reduces the appearance of wrinkles generally after the first injection. However, the effects of Botox injections last less than a year. Additionally, Botox injections require administration by a medical professional and should not be self-administered. This administration by a medical professional makes Botox injections costly and time consuming. Furthermore, Botox injections have been known to cause serious side-effects if improperly administered.

Additionally, some cosmetic creams, gels, or ointments claim to repair wrinkled skin. However, these products assert that they provide a long-term fix to the appearance of wrinkles after repeated use.

Thus, there is nothing on the market that provides a near immediate softening of wrinkles suitable for daily wear. Therefore, a need exists to improve upon the cosmetic products in the market.

### SUMMARY AND OBJECTS OF THE INVENTION

The disclosed invention addresses the above-identified issues and improves upon the prior art by providing a safe daily wear cosmetic essence that temporarily softens wrinkles and which may be worn in public. The cosmetic essence may be applied to a wrinkled area of skin, such as on the eleven-lines, by gently applying the essence onto the skin. The essence then causes a nearly immediate tightening effect on the skin reducing the appearance of wrinkles. Furthermore, after the essence is applied to the skin, the essence is invisible and scentless, so it is intended to be worn in public.

When applied to skin, the cosmetic essence causes a tightening effect. This tightening effect is what may reduce the appearance of wrinkles. As the skin becomes more taught, wrinkles lighten and flatten to become less visible. Therefore, the cosmetic essence may be applied to the eleven-lines to reduce the visibility of the eleven-lines.

The essence is easily applied and may be self-administered. A user may first clean the area of skin where the essence will be applied. The user may then gently spread a dab of the essence onto the skin. The essence is then smoothed into a very thin layer over the wrinkled area. As the essence dries, the eleven-lines flatten for the desired effect.

Once applied, the cosmetic essence is intended to be worn in public. The essence is invisible after it is applied onto the skin. Furthermore, the essence is scentless. Therefore, it is not noticeable when a user has applied the essence. Thus, the essence is well-suited to be worn in public.

Furthermore, the essence's near immediate effect makes the essence well-suited for daily wear. The essence may produce the tightening effect nearly immediately after application. A user does not need to wait for results after repeated uses. The essence may soften the appearance of wrinkles after a first use. Thus, a user may use the essence daily to soften the appearance of wrinkles each day.

The softening of wrinkles may only last for a limited duration of time. The tightening effect may substantially wear off after approximately eight (8) hours. Further, the essence may be washed off using only water. Thus, the essence may be applied to a wrinkled area, such as the eleven-lines, at the beginning of a day or before an event. The essence's tightening effect may soften the wrinkles throughout the day or event. Then, at the end of the day or event when the tightening effect may have begun to wear off, the essence may be completely washed off. The essence may then be reapplied the next day or prior to another event.

Additionally, the cosmetic essence may prevent the user from frowning. Because the eleven-lines may become more prominent when a person frowns, the tightening effect of the essence may be felt more when a user frowns. This may act as a preventive measure causing the user to avoid frowning, which may subsequently prevent the deepening of the eleven-lines.

The formula for the cosmetic essence may include a principal component and a supplementary component. To produce the tightening effect, the principal component may act as a tightening agent. Further, the principal component may constitute a majority percentage of the essence. The supplementary components may include a preserving agent, a thickening agent, and an anti-flaking agent.

In one embodiment, the principal agent is albumen, for example egg white. Albumen is predominantly made up of water and proteins. The primary protein in albumin is ovalbumin. In this embodiment, the albumen acts as the tightening agent.

The supplementary components are required to be mixed with the principal component to make the essence usable. The supplementary components may constitute a small percentage of the essence. In one embodiment, the supplementary components may make up less than 3% of the essence.

The supplementary components may include a preserving agent. The preserving agent may act to keep the albumen from spoiling. In one embodiment, the preserving agent may be one or more salts, such as potassium sorbate and sodium benzoate. These salts may constitute approximately 1% of the essence.

Additionally, the supplementary components may include a thickening agent. The thickening agent may be added to alter the consistency of the essence. Because albumen is predominantly water, a thickening agent may be included to prevent the essence from being too watery or runny. In one embodiment, the thickening agent is one or more gum additives, such as guar gum or xanthan gum. The gum additives may constitute appropriately 1% of the essence.

Further, the supplementary components may include an anti-flaking agent. The anti-flaking agent may be added to allow the essence to be effectively applied to the skin. Without an anti-flaking agent, the essence may begin to flake when being applied and may not be adequately absorbed by the skin. In one embodiment, the anti-flaking agent is one or more powders, such as aloe vera powder or pearl powder. These powders may constitute approximately 0.6% of the essence.

These and other aspects and objects of the present invention will be better appreciated and understood when considered in conjunction with the following description and the accompanying drawings. It should be understood, however, that the following description, while indicating preferred embodiments of the present invention, is given by way of illustration and not of limitation. Many changes and modifications may be made within the scope of the present invention without departing from the spirit thereof, and the invention includes all such modifications.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Preferred exemplary embodiments of the invention are illustrated in the accompanying drawings in which like reference numerals represent like parts throughout, and in which:

FIG. 1 illustrates one formula embodiment making up a cosmetic essence of the present invention.

FIG. 2 illustrates a flowchart of a method of using a cosmetic essence of the present invention to soften wrinkles.

FIG. 3 illustrates the eleven-lines between the eyebrows.

FIG. 3A illustrates the eleven-lines prior to the application of the cosmetic essence.

FIG. 3B illustrates the eleven-lines after the application of the cosmetic essence.

In describing the preferred embodiment of the invention that is illustrated in the drawings, specific terminology will be resorted to for the sake of clarity. However, it is not intended that the invention be limited to the specific terms so selected, and it is to be understood that each specific term includes all technical equivalents, which operate in a similar manner to accomplish a similar purpose. For example, the word connected, attached, or terms similar thereto are often used. They are not limited to direct connection but include connection through other elements where such connection is recognized as being equivalent by those skilled in the art.

### DETAILED DESCRIPTION OF THE INVENTION

The present invention and the various features and advantageous details thereof are explained more fully with reference to the non-limiting embodiments described in detail in the following description.

The present invention provides a daily wear cosmetic essence that temporarily softens wrinkles. The essence may be easily self-applied to a wrinkled area of skin, such as the eleven-lines 1. When applied, the essence is invisible and scentless, such that it is well suited to be worn in public. The essence causes a nearly immediate tightening effect in the area to which it is applied that lightens and flattens wrinkles. This tightening effect is temporary, and the essence may be washed off with just water.

As used herein, the term “soften” or “softening” of wrinkles refers to reducing the appearance of, lightening, or flattening wrinkles or the equivalent thereof, such that wrinkles may be less noticeable. Additionally, the term “daily wear” refers to the repeated use of a product every day in regular occurrences. Further, it is understood that the term “essence” may include any cream, gel, or ointment or equivalent thereof.

### Method of Use

The present cosmetic essence improves upon the prior art in variety of ways. The essence may be easily applied within minutes by gently applying the essence onto a wrinkled area of skin. The essence may be self-applied opposed to needing a medical professional administer a treatment. Additionally, when applied, the essence may be unnoticeable because of its clear and scentless features. Therefore, the essence is suitable to be worn in public as opposed to other treatments, which are readily noticeable when applied and designed to be used in private settings. Further, the essence may soften wrinkles nearly immediately after being applied. This is in contrast to other treatments that require repeated use in an attempt to repair wrinkled areas of skin. These benefits of the present invention make the essence well suited for daily wear.

A primary feature of the essence is the tightening effect caused after the essence is applied. Once the essence is applied to a wrinkled area of skin, such as the eleven-lines 1, the essence causes a tightening effect. The tightening effect stretches the wrinkled skin softening the appearance of wrinkles. The tightening effect may cause the wrinkled skin to become tauter, reducing the visibility of wrinkles.

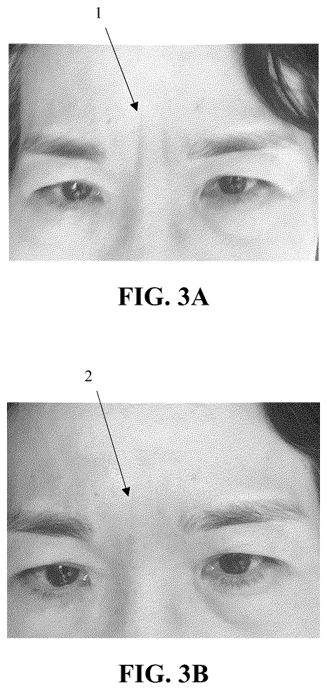

As evidenced in FIGS. 3A and 3B, the essence may reduce the visibility of wrinkles, such as the eleven-lines 1. FIG. 3A shows the eleven-lines 1 without the cosmetic essence being applied. In comparison, FIG. 3B shows softened eleven-lines 2 after the essence is applied. The softened eleven-lines 2 after the essence is applied appear less visible than the eleven-lines 1 before the essence was applied.

The cosmetic essence may be easily self-applied. The essence may be applied by taking a dab of the essence and placing it on a wrinkled area of skin, such as the eleven-lines 1. The essence may then be spread over the wrinkled area and gently smoothed onto the skin, as is done with most typical cosmetic creams, gels, or ointments. The essence may dry on and be absorbed by the skin. Furthermore, the essence may be more effectively applied and produce better results if the area of application is washed and dried prior to being applied. The simplicity of the application process may allow a user to easily apply the essence on their own without the need of any additional help. This self-applying process is cheap and easy, making the essence well suited for daily wear.

Additionally, the present essence is well suited for daily wear because the essence may be designed to be worn in public. The essence is clear and odorless. Therefore, after the essence is applied, the essence may be invisible and scentless. Another person may be unlikely to notice when the essence has been applied on a user making the essence well suited to be worn in public.

Further, the cosmetic essence softens wrinkles nearly immediately after the first application. The essence may cause a tightening effect that may soften wrinkles after every application. The cosmetic essence does not require repeated use, but rather may produce results right away.

The essence's tightening effect only lasts for a temporary duration of time. While the essence is applied to a wrinkled area of skin, the essence slowly evaporates. Therefore, after a temporary period of time, the essence will be substantially evaporated resulting in the loss of the tightening effect. The tightening effect may substantially wear off approximately one day after application. Further, the tightening effect may substantially wear off approximately eight (8) hours after application. Additionally, the cosmetic essence may be easily removed as the essence may be easily washed off with just water.

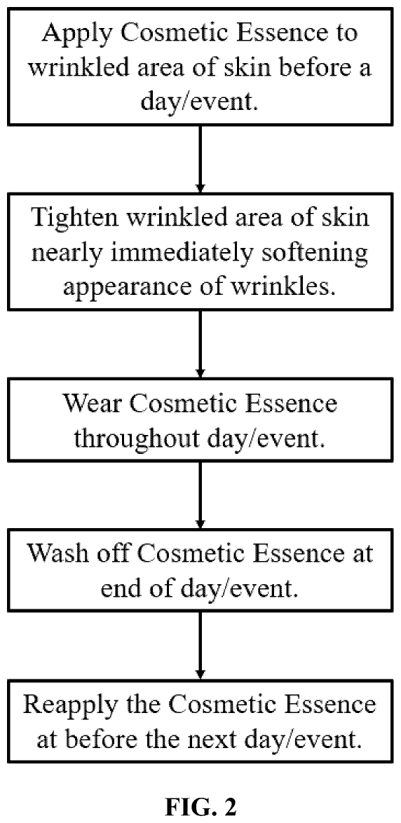

These benefits provided by the essence make the essence well suited for daily wear, as shown FIG. 2. A user may easily self-apply the essence to wrinkled skin, such as the eleven-lines 1, at the beginning of the day or before an event. The tightening effect may act nearly immediately softening the appearance of wrinkles. Because the essence may be invisible and scentless when applied, the essence may be worn throughout a day or a public event without others noticing that the essence is being used. Then, at the end of the day or event, the essence and the remaining tightening effect may be easily washed off the face. Further, the essence may then be reapplied at the beginning of the next day or before another event.

As previously stated, one wrinkled area to which the essence may be applied is the eleven-lines 1. The eleven-lines 1 are two or more vertical wrinkles between the eyebrows that may extend from the forehead to the eyes, as shown in FIG. 3A. Similar to most wrinkles, the eleven-lines 1 may become more pronounced with age, making a person wish to soften the appearance of the eleven-lines 1. However, in contrast with other facial wrinkles, the eleven-lines 1 may crease and become more pronounced when a person frowns.

The tightening effect of the essence may additionally serve as a reminder to a user to avoid frowning. If the essence is applied to the eleven-lines 1, the essence's tightening effect may provide resistance when the user frowns. This resistance may be felt by the user. This feeling of resistance may be used as a signal to the user that she/he is frowning, possibly serving to deter such a facial configuration. Further, a repeated frowning deterrence may reduce the number of times a user's eleven-lines 1 crease and become more prominent. Reducing this repeated creasing may help prevent the deepening of the eleven-lines 1.

### Essence Formula

The formula for the cosmetic essence may include a principal component and supplementary components. The principal component may act as a tightening agent causing the tightening effect. Additionally, the principal component may make up a majority percentage of the essence. Further, the principal component may make up 97.4% of the essence. The supplementary components may include a preserving agent, a thickening agent, and an anti-flaking agent. Further, the supplementary components may make up 2.6% of the essence.

The principal component may be albumen, for example egg white. Albumen is predominantly made up of water. Albumen also includes a variety of proteins, the predominant protein being ovalbumin. The coagulating properties of the albumen may cause the tightening effect. The source of the albumen may be a chicken egg. Other eggs, such as duck or quail, may also be the source of the albumen.

As for the supplementary components, one supplementary component may be a preserving agent. The principal component, such as albumen, may have a tendency to spoil having adverse effects on the essence. The preserving agent may be added to the essence to prolong the life of the principal component before it spoils. The preserving agent may constitute approximately 1% of the essence. Additionally, the preserving agent may include one or more salts. Further, the preserving agent may include potassium sorbate and sodium benzoate.

Another supplementary component may be a thickening agent. A principal component such as albumen is predominantly composed of water. An essence with such a high percentage of water may be too viscous. For example, the essence may be runny making it difficult to apply. A thickening agent may decrease the viscosity of the essence, making the essence easier to handle and apply. The thickening agent may constitute approximately 1% of the essence. Additionally, the thickening agent may include one or more gum additives. Further, the thickening agent may include guar gum or xanthan gum.

An additional supplementary component may be an anti-flaking agent. Rather than drying on or being absorbed by the skin, a principal component such as albumen may begin to flake when applied. Not only may the flaking inhibit the tightening effect, but the flaking may also make the essence visible to others. The anti-flaking agent may prevent this flaking. The anti-flaking agent may constitute approximately 0.6% of the essence. Additionally, the anti-flaking agent may include one or more powders. Further, the anti-flaking agent may include aloe vera powder and pearl powder.

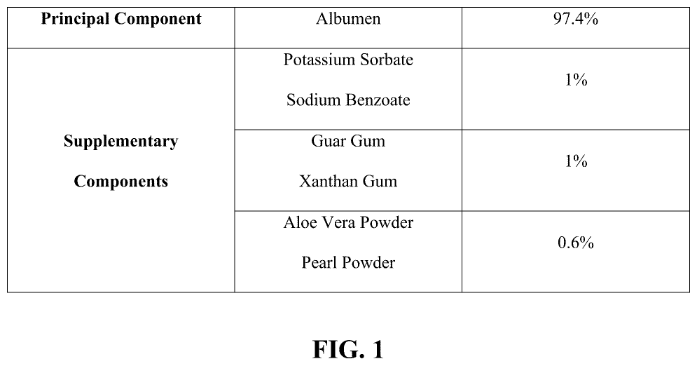

As shown in FIG. 1, the essence may include a principal agent, such as albumen, that acts as a tightening agent and constitutes a majority percentage of the essence. Furthermore, the essence may include supplementary components constituting approximately 2.6% of the essence. The supplementary components may include (i) a preserving agent, such as potassium sorbate and sodium benzoate, (ii) a thickening agent, such as guar gum and xanthan gum, and (iii) an anti-flaking agent, such as aloe vera powder and pearl powder. A small variation of one component may dramatically alter the effectiveness of the essence. A significant amount of trial and error was committed to forming the embodiment present in this paragraph.

The components for the essence may be natural and food-grade materials. However, the same effects may be able to be produced by providing non-natural or non-food grade components.

The cosmetic essence is made by mixing all the components together. The supplementary components may be added to the principal component, such as albumen. The components may then be mixed until all the supplementary components are dissolved in the principal component. The components must be thoroughly mixed immediately after guar gum may be added. Guar gum may not adequately dissolve if the components are not sufficiently mixed within the first three (3) minutes of adding the guar gum.

Once the essence is mixed, the essence may be stored in a glass container. The glass container may be pasteurized prior to storage by being placed in boiling water for approximately ten (10) minutes. The essence may be used immediately. However, the essence may provide better results if waiting at least 24 hours before being applied. Further, the essence may be mixed and subsequently stored at room temperature. The as a finalized product may have a low viscosity, be clear, and have no smell.

Although the best mode contemplated by the inventors of carrying out the present invention is disclosed above, practice of the present invention is not limited thereto. It will be manifest that various additions, modifications and rearrangements of the features of the present invention may be made without deviating from the spirit and scope of the underlying inventive concept.

Moreover, the individual components need not be formed in the disclosed shapes, or assembled in the disclosed configuration, but could be provided in virtually any shape, and assembled in virtually any configuration. Furthermore, all the disclosed features of each disclosed embodiment can be combined with, or substituted for, the disclosed features of every other disclosed embodiment except where such features are mutually exclusive.

1. A method of temporarily softening an appearance of wrinkles, comprising the steps of:
providing a cosmetic essence, the cosmetic essence being clear;
spreading the cosmetic essence onto a wrinkled area of skin;
invisibly wearing the cosmetic essence on the wrinkled area of skin;
causing a tightening effect with the cosmetic essence; and
softening the appearance of wrinkles with the tightening effect for a temporary duration of time not greater than one day.

2. The method of claim 1, further comprising:
wearing the cosmetic essence on the wrinkled area of skin for at least eight hours;
washing the cosmetic essence off the wrinkled area of skin;
stopping the tightening effect; and
reapplying the cosmetic essence onto the wrinkled area of skin to cause the tightening effect.

3. The method of claim 2, further comprising:
applying the cosmetic essence onto the wrinkled area of skin at the beginning of a first day;
washing the cosmetic essence off the wrinkled area of skin at the end of a first day; and
reapplying the cosmetic essence onto the wrinkled area of skin at the beginning of a second day.

4. The method of claim 1, wherein the wrinkled area of skin is on a human face.

5. The method of claim 4, wherein the wrinkled area of skin extends from a forehead to at least an eyebrow.

6. The method of claim 1, wherein causing the tightening effect with the cosmetic essence occurs nearly immediately after applying the cosmetic essence onto a wrinkled area of skin.

7. A method of softening an appearance of wrinkles, comprising the acts of:
providing a cosmetic essence, the cosmetic essence comprises
(i) a tightening agent comprising albumen, and
(ii) supplementary components consisting of at least one salt, at least one gum additive, and at least one powder, wherein the supplementary components constitute less than 3% of the cosmetic essence;
spreading the cosmetic essence on a wrinkled area of skin;
tightening the wrinkled area of skin; and
reducing the appearance of wrinkles in the wrinkled area of skin for a temporary duration of time.

8. The method of claim 7, further comprising:
wearing the cosmetic essence on the wrinkled area of skin in public for the temporary duration of time, wherein the temporary duration of time has a beginning and an end;
removing the cosmetic essence from the wrinkled area of skin at the end of the temporary duration of time; and
reapplying the cosmetic essence on the wrinkled area of skin.

9. The method of claim 8, further comprising:
cleaning the wrinkled area of skin prior to applying the cosmetic essence.

10. The method of claim 7, wherein:
the tightening agent is albumen and constitutes a majority percentage of the cosmetic essence.

11. The method of claim 10, wherein:
the salt constitutes approximately 1% of the cosmetic essence, the gum additive constitutes approximately 1% of the cosmetic essence, and the powder constitutes approximately 0.6% of the cosmetic essence.

12. The method of claim 711, wherein:
the salt is selected from a group consisting of potassium sorbate, sodium benzoate, and combinations thereof;
the gum additive is selected from a group consisting of guar gum, xanthan gum, and combinations thereof; and
the powder is selected from a group consisting of aloe vera powder, pearl powder, and combinations thereof.

13. The method of claim 12, wherein the wrinkled area of skin is on a human face.

14. The method of claim 13, wherein the wrinkled area of skin extends from a forehead to at least an eyebrow.

15. The method of claim 7, wherein tightening the wrinkled area of skin occurs nearly immediately after applying the cosmetic essence to a wrinkled area of skin.

16. A cosmetic essence comprising:
a principal component that constitutes a majority percentage for the cosmetic essence, wherein the principal component is albumen and acts as a skin tightening agent; and
supplementary components constituting less than 3% of the cosmetic essence, the supplementary components consisting of at least one salt, at least one gum additive, and at least one powder.

17. (canceled)

18. The cosmetic essence of claim 17, wherein:
the supplementary components constitute approximately 2.6% of the cosmetic essence, wherein:
the salt constitutes approximately 1% of the cosmetic essence;
the gum additive constitutes approximately 1% of the cosmetic essence; and
the powder constitutes approximately 0.6% of the cosmetic essence.

19. The cosmetic essence of claim 18, wherein:
the salt consists of potassium sorbate, sodium benzoate, and combinations thereof;
the gum additive consists of guar gum, xanthan gum, and combinations thereof; and
the powder consists of aloe vera powder, and pearl powder, and combinations thereof.

20. The cosmetic essence of claim 16, wherein:
the principal component contains ovalbumin proteins.

21. The cosmetic essence of claim 16, wherein the cosmetic essence consists essentially of
the principal component consisting of albumen; and
the supplementary components, wherein
(i) the salt consists of potassium sorbate, and sodium benzoate, and combinations thereof,
(ii) the gum additive consists of guar gum, and xanthan gum, and combinations thereof, and
(iii) the powder consists of aloe vera powder, and pearl powder, and combinations thereof.
